(19) United States
(12) Patent Application Publication
(10) Pub. No.: US 2003/0143264 A1
(43) Pub. Date: Jul. 31, 2003

(54) Topical anesthetic-antiseptic patch

(76) Inventor: Vincent Margiotta, Englewood Cliffs, NJ (US)

Correspondence Address: Vincent J. Margiotta, 43 Geraldine Rd., Englewood Cliffs, NJ, 07632, US

(21) Appl. No.: 10/058,373

(22) Filed: Jan. 30, 2002

(51) Int. Cl. A61L 15/16

(52) U.S. Cl. 424/446

(57) ABSTRACT

A pre-vaccination or pre-procedural topical anesthetic/antiseptic patch which functions not unlike a nicotine patch and which is essentially an adhesive bandage containing a topical anesthetic and a topical antiseptic. The bandage anesthetic/antiseptic carrying device may be of any shape or size. It is applied to a person's skin at a site where a needle is to be inserted such that the site will have a topical anesthetic and a topical antiseptic applied. When the bandage is removed from the site prior to a needle being inserted a visible delineation of a target area is visible on the skin. That is, that side of the bandage which contacts the skin will contain a demarcating fabric material, in the form of a circle or any other geometric shape, smaller than the diameter or size of the adhesive bandage, that will remain on the wearer's skin whereby when the bandage is removed the demarcating fabric material will delineate the site which is the “target” area. Particularly important is the fact that once the bandage or patch is removed, prior to a needle being inserted, the target area or demarcated area of any or geometric shape, defined by the demarcating fabric material made of possibly but not limited to plastic or latex or cotton will be left on the skin which clearly delineates where the anesthetic/antiseptic is and therefore, where the needle should be inserted.

### BACKGROUND OF INVENTION

1. Field of the Invention

The present invention generally involves the field of technology pertaining to an anesthetic and antiseptic patch, principally used for infants and small children, but which may be used by others, wherein the device is essentially a common, over the counter, preferably round adhesive bandage containing a topical anesthetic and a topical antiseptic. The device is placed on a person's skin at a site where a needle is to be inserted such that the area will have a topical anesthetic applied for anesthetic effect. Additionally the device will have the effect of disinfecting said skin area as a function of the topical antiseptic. Obviously, the device is applied to the skin in advance of needle insertion. It should be emphasized that both the adhesive bandage or any other type of bandage material, as well as the anesthetic agents and antiseptics, are well known and are presently approved for over the counter sale.

It should be obvious that the anesthetic and antiseptic carrying material may be something other than an adhesive bandage, but can be of other material that will not only carry the anesthetic and antiseptic, but will also adhere to a person's skin.

For purposes of further discussing this device, the adhesive bandage or skin contacting anesthetic-antiseptic solution carrying material will be referred to as an adhesive patch. It is this adhesive patch that will carry any type of topical anesthetic and topical antiseptic that will prove satisfactory for this purpose and will also carry a natural or synthetic fabric material entity, possibly made of, but not limited to latex or plastic or cotton, that will remain on the wearer's skin hereon referred to as a demarcating fabric material, after the patch is removed for the purpose of demarcating the area that has been treated with the topical agents. When the adhesive patch is removed, prior to a needle being inserted, it will therefore leave a visible delineation of a “target” area which may be of any geometric shape, defined by the demarcating fabric material that will remain on the wearer's skin, preferably circular in shape, which clearly marks the site where a needle, etc. is to be inserted.

The adhesive patch further comprises a pad of absorbent material, such as but not limited to cotton, which contains both active and inactive ingredients. That portion of the absorbent pad to be placed over the site to be injected or punctured will be somewhat smaller in size than the overall size of the adhesive patch.

It is anticipated that the active ingredients indicated above will be benzocaine or lidocaine functioning as the active topical anesthetic ingredient, benzalkonium chloride or benzethonium chloride as the topical antiseptic ingredient, and the inactive ingredient indicated above will be glycerin or purified water or any other benign vehicle. These ingredients can be in varying proportions depending upon the age of the recipient.

Any well-known synthetic or natural fabric material may be used on the skin side of the adhesive patch to mark the spot to be penetrated. It should also be obvious that the active and inactive ingredients can be other than those mentioned above.

2. Description of the Prior Art

A search of the prior art has uncovered the following patents: U.S. Patent to Devillez (U.S. Pat. No. 5,641,507) and Vishnupad, et al (U.S. Pat. No. 4,992,508) and Margiotta (U.S. Pat. No. 6,274,167).

Devillez does not teach a fabric material that remains on the skin for the purpose of demarcation, once the patch is removed. Vishnupad does not mention any material that remains on the wearer's skin for the purpose of demarcating the area that has been treated with the active agents. There is no teaching here that when removing the “casts” of Vishnupad from a person's skin that it would therefore leave “. . . a targeted area for reception of a needle.” Margiotta does reference a “dye operatively associated with said pad; whereby, when said patch is applied to a person's skin and then removed, the skin is anesthetized and a target area for reception of a needle is delineated.” Clearly the use of a fabric material such as but not limited to plastic or latex to demarcate and/or circumscribe the target area is different than a “dye”. No mention is made in any of these references of a material such as a plastic or latex other type of material entity that will remain on the wearer's skin for the purpose of demarcating the treated area. There is no teaching in these references of a circumscribing/demarcating material remnant such as but not limited to a plastic or latex that remains on the wearer's skin after the patch is removed for the purpose of demarcating the treated area.

There is no teaching in either Devillez or Vishnupad specifying the production of a demarcated area that remains as an artifact on the skin, once said device is removed from the wearer, for the purpose of delineation and demarcation of the treated area. Margiotta specifies a demarcation that is produced by a dye, but clearly does not incorporate the use of a fabric material entity such as plastic or latex or cotton to demarcate the area that has been treated with the active agents once the patch has been removed from the wearer's skin.

Additionally, it is clear neither of these (Devillez and Vishnupad) teach the production of a combined delineated, anesthetized and disinfected area of skin into which a needle will be inserted.

The fact that the herein presented device produces a visible demarcation using a circumscribing/demarcating fabric material remnant consisting of, but not limited to plastic or latex as an artifact on the skin of the wearer after its removal for the purpose of delineating the affected site, makes it unique and novel.

### SUMMARY OF INVENTION

According to the present invention there is provided an adhesive patch, not unlike current nicotine patches, comprising an adhesive bandage backing, preferably of plastic, but possibly of other materials, which serves as the backing for the adhesive patch for this invention. On that side of the adhesive patch is a bandage of gauze or cotton or any other suitable absorbent material which adheres to the adhesive bandage or patch by any suitable means.

One side of the gauze pad or cotton pad may be impermeable which is the side that adheres to the adhesive bandage backing. The other side of the pad, that is, the side which will contact the skin, is absorbent.

A quantity of a solution containing a topical anesthetic such as, but not limited to, benzocaine, and a topical antiseptic such as, but not limited to, benzalkonium chloride, is then applied to the absorbent pad and surrounding that sample or supply of above mentioned solution is a circumscribing/demarcating synthetic or natural fabric material entity that will remain on the wearer's skin after the patch is removed for the purpose of demarcating the treated area in a preferred embodiment it is that circular shaped the inside of which forms the target area for a needle.

In the preferred embodiment when the adhesive bandage is removed it will leave a demarcated target area defined by the circumscribing/demarcating fabric material entity, that has been exposed to both a topical anesthetic and a topical antiseptic for needle insertion.

For the preferred embodiment the entire device is protected with a plastic or foil strip such that the adhesive patch is kept moist at all times. The entire device is packaged as sterile, as are other adhesive bandages.

It is therefore an object of the present invention to provide an anesthetic/antiseptic patch principally used for but not limited to infants and small children.

It is another object of the invention to provide an anesthetic/antiseptic patch which is an adhesive patch to be placed on a person's skin at a site where a needle is to be inserted.

It is a further object of the invention to provide such a patch comprising a pad of absorbent material such as cotton containing a topical anesthetic and topical antiseptic comprising both active and inactive ingredients.

It is yet a further object of the invention to provide such a patch wherein the active ingredients are benzocaine topical anesthetic and benzalkonium chloride topical antiseptic and the inactive ingredients are glycerin and/or purified water.

It is still a further object of the invention to provide such a patch wherein the active and inactive ingredients may be of varying proportions depending upon the age of the recipient and the degree of desired functional effect.

It is still a further object of the invention to provide such a patch wherein a skin side of the patch further contains any suitable synthetic or natural fabric material that will remain as a demarcating remnant after the patch is removed so that the site on the skin to be penetrated with the needle is defined.

It is still a further object of the invention to provide such a patch wherein a quantity of a solution containing topical anesthetic and topical antiseptic is applied to the absorbent pad and surrounding that quantity of said solution is the circumscribing/demarcating fabric material remnant, preferably of circular shape.

These and further objects, features and advantages of the invention shall become apparent from the following detailed description of a preferred embodiment thereof when taken in conjunction with the professionally drafted drawings wherein like reference characters refer to corresponding parts in the several views.

### BRIEF DESCRIPTION OF THE DRAWINGS

FIG. 1 is a plan view of the patch showing the non-skin contacting side of the adhesive patch.

FIG. 2 is a view showing the reverse side, which is the skin contacting side.

FIG. 3 is a cross sectional view taken on line 3-3 of FIG. 2.

FIG. 4 is a view similar to FIG. 2 but showing demarcating fabric material surrounding a pad of absorbent material.

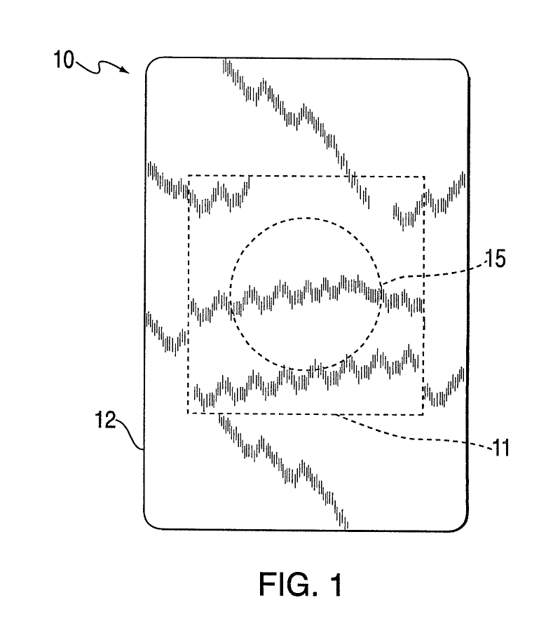

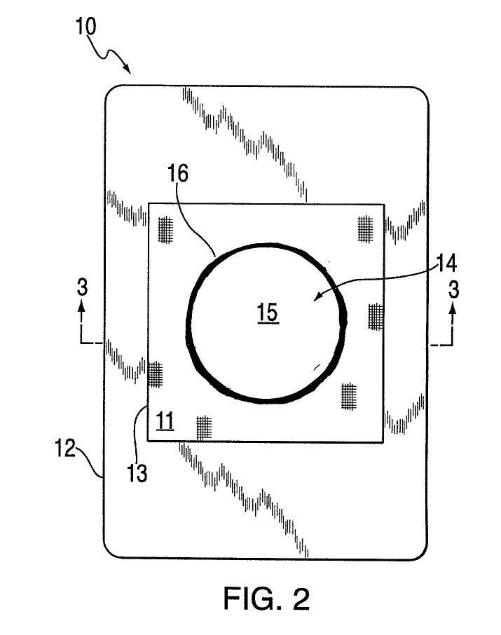

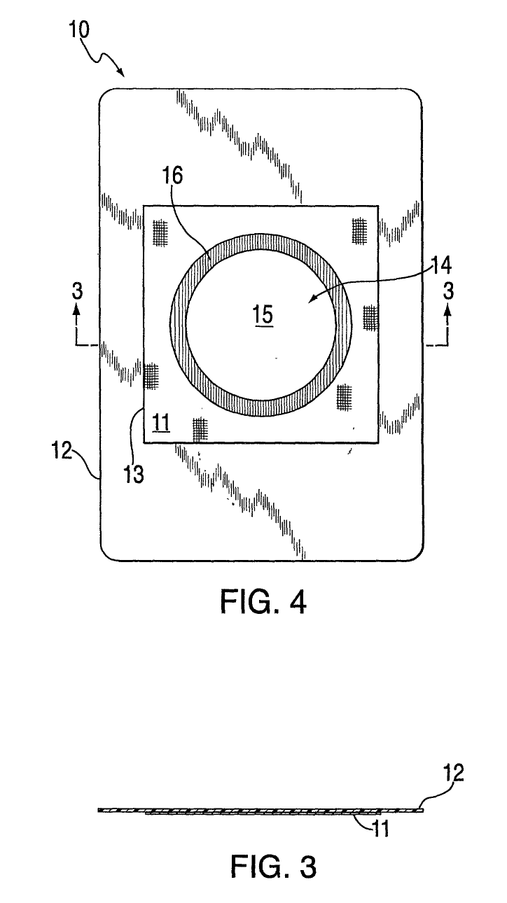

### DETAILED DESCRIPTION OF THE PREFERRED EMBODIMENT

An anesthetic/antiseptic adhesive patch which may be applied to a persons skin by anyone including the person themselves, a parent, pediatrician, medical auxiliary, etc. at the site where a needle is to be inserted, for any reason, and which contains a topical anesthetic and a topical antiseptic solution carried by an absorbent material, such as cotton, and which contains both active and inactive ingredients for the purpose of applying a topical anesthetic and a topical antiseptic to the skin will now be described with reference to the drawings.

The adhesive patch is shown generally at 10, having a gauze pad 11, attached to one side of a backing 12, preferably of plastic, and wherein the gauze pad can have an impermeable side 13, coacting with the backing 12, and an absorbent top side 14, which coacts the wearer's skin.

A solution containing a topical anesthetic and a topical antiseptic 15, is applied to gauze pad 11, and, surrounding that portion 15, of gauze pad 11, containing the solution, is a synthetic or natural fabric material entity designed to remain on the wearer's skin, in using the adhesive patch 10, as will be more fully described below, will leave a demarcation zone or target area on the skin when the adhesive patch is removed. This target area is where a needle will be inserted.

The demarcating fabric material entity is applied to the gauze pad by any suitable means and, while shown as circular in the drawings, may be of any other convenient geometric shape leaving an exposed center.

The entire patch described above is preferably covered with a plastic or foil strip to retain moisture and retard evaporation. The entire patch is packaged as sterile, as are other over-the-counter adhesive bandages.

The anesthetic/antiseptic solution, 15, shown on the gauze pad, 11, comprises well-known ingredients including benzocaine and/or lidocaine, benzalkonium chloride and/or benzethonium chloride, polyethylene glycol/glycerin and/or purified water, and possibly sorbitol as a preservative.

It should be understood that the various concentrations of both or either of the active or inactive ingredients can be varied to alter duration or intensity or other affectable properties.

In addition to the preferred embodiments discussed above, there are two other embodiments well within the contemplation of the invention and shown in the drawings and generally discussed above. Both of these other embodiments comprise the backing, 12, gauze pad, 11, and the demarcating fabric material entity, 16. Still added to the gauze pad, 11, is the anesthetic/antiseptic solution generally shown at 15.

In operation, the adhesive patch is removed from its protective device such as the plastic or foil strip, and is placed on a person's skin, at a predetermined time, and most likely a predetermined site, with the absorbent gauze pad contacting the skin. At this time, the skin area under the gauze pad will come into contact with the topical anesthetic/antiseptic solution. When a needle is to be inserted into the person, at the site from which the patch was removed leaving a demarcated or target area defined by the demarcating fabric material, which remains on the wearer's skin after the patch is removed, of any geometric shape, thereby indicating the site for the needle to be inserted. When the patch is removed from the skin it is disposed of. After the needle is inserted and/or withdrawn from the demarcated area the demarcating fabric material may be removed.

Though the invention has been described and illustrated with reference to a preferred embodiment thereof, those skilled in the art will appreciate that various changes and modifications in shape, size, composition and arrangement of parts may be resorted to without departing form the spirit of the invention or scope of subjoined claims.

What is claimed:

1. An anesthetic and antiseptic carrying adhesive patch attachable to a person's skin comprising:
(a) A backing;
(b) A pad attached to one side of said backing;
(c) A solution applied to said pad containing topical anesthetic and antiseptic; and,
(d) Natural or synthetic or blended natural and synthetic fabric material entity operatively associated with said pad;
whereby, when said patch is applied to a person's skin and then removed, the skin is anesthetized and treated with an antiseptic, and a target area for reception of a needle is delineated by said fabric material entity.

2. The anesthetic and antiseptic carrying adhesive patch of claim 1, wherein said backing is plastic.

3. The anesthetic and antiseptic carrying adhesive patch of claim 2 wherein said pad is gauze.

4. The anesthetic and antiseptic carrying adhesive patch of claim 3 wherein said solution is made of benzocaine and benzalkonium chloride.

5. The anesthetic and antiseptic carrying adhesive patch of claim 4 wherein said anesthetic and antiseptic solution contains both active and inactive solutions.

6. The anesthetic and antiseptic carrying adhesive patch of claim 5 wherein said gauze pad has an impermeable side coacting with said backing and an absorbent top side which coacts with a person's skin.

7. The anesthetic carrying adhesive patch of claim 6 wherein said benzocaine and benzalkonium chloride and said dye are mixed together and applied to said gauze pad whereby when said adhesive patch is removed from a person's skin a target area is left on anesthetized and disinfected skin.

8. The anesthetic and antiseptic carrying adhesive patch of claim 7 wherein said benzocaine/benzalkonium chloride solution comprises benzocaine and benzalkonium chloride, polyethylene glycol, purified water, and sorbitol preservative.
